(19) United States
(12) Patent Application Publication
(10) Pub. No.: US 2016/0081915 A1
(43) Pub. Date: Mar. 24, 2016

(54) TRANSDERMAL FORMULATIONS OF FLUTICASONE

(71) Applicant: STRATEGIC SCIENCE & TECHNOLOGIES, LLC, Cambridge, MA (US)

(72) Inventors: Elias James Corey, Cambridge, MA (US); Eric T. Fossel, Cambridge, MA (US)

(73) Assignee: Strategic Science & Technologies, LLC, Cambridge, MA (US)

(21) Appl. No.: 14/773,586

(22) PCT Filed: Mar. 14, 2014
(86) PCT No.: PCT/US2014/027157

§ 371 Date: Sep. 8, 2015

Related U.S. Application Data
(60) Provisional application No. 61/790,770, filed on Mar. 15, 2013.

(51) Int. Cl. A61K 9/00; A61K 31/198; A61K 47/02; A61K 47/12; A61K 47/06; A61K 47/26; A61K 9/06; A61K 47/14; A61K 47/10; A61K 31/569; A61K 47/36

(52) U.S. Cl. CPC A61K 9/0014; A61K 31/569; A61K 31/198; A61K 47/02; A61K 47/12; A61K 47/36; A61K 47/26; A61K 9/06; A61K 47/14; A61K 47/10; A61K 47/06

(57) ABSTRACT

The present invention generally relates to the transdermal delivery of fluticasone and other glucocorticoids. In some aspects, transdermal delivery may be facilitated by the use of a hostile biophysical environment, for example, a high ionic strength environment. One set of embodiments provides a composition for transdermal delivery comprising fluticasone and/or a salt thereof, and optionally, a nitric oxide donor. Other glucocorticoids can also be used in some cases. Such compositions may be used to facilitate the delivery of effective amounts of fluticasone or other glucocorticoids systemically or to deeper tissues, rather than only locally or superficially, and in some cases, the effects of fluticasone or other glucocorticoids may be prolonged for unexpectedly long periods of time, e.g., days or weeks.

### RELATED APPLICATIONS

This application claims the benefit of U.S. Provisional Patent Application Ser. No. 61/790,770, filed Mar. 15, 2013, entitled “Topical Formulation of Fluticasone,” incorporated herein by reference in its entirety.

### FIELD OF INVENTION

The present invention generally relates to the transdermal delivery of fluticasone and other glucocorticoids.

### BACKGROUND

Fluticasone is a steroidal anti-inflammatory agent that is used principally for asthma. In addition, it has been considered also for topical use in the treatment of itching or inflammation. Fluticasone is also used intranasally in the treatment of allergic rhinitis and other inflammatory nasal conditions and of nasal polyps, and by inhalation in the treatment of asthma, e.g., as an aerosol. It is believed that fluticasone acts to decrease inflammation by inhibiting eicosanoid (e.g., prostaglandin) synthesis. Fluticasone may also inhibit the accumulation of macrophages and leukocytes at sites of inflammation. In addition, fluticasone may inhibit phagocytosis and lysosomal enzyme release. Typically, fluticasone is applied topically twice a day, e.g., to a site of inflammation.

### SUMMARY OF THE INVENTION

The present invention generally relates to the transdermal delivery of fluticasone and other glucocorticoids. The subject matter of the present invention involves, in some cases, interrelated products, alternative solutions to a particular problem, and/or a plurality of different uses of one or more systems and/or articles.

In some embodiments, the use of rapidly metabolized glucocorticoids is especially advantageous for safety reasons.

In accordance with one aspect, the present invention is generally directed to a composition for transdermal delivery. While others have suggested delivering fluticasone topically in creams, such formulations have generally been limited to the treatment of skin conditions. However, certain aspects of the present invention surprisingly allow formulations that improve the effectiveness of fluticasone, for instance, for prolonged periods of time (e.g., days or weeks instead of hours). In addition, in some cases, certain conditions may be treated using formulations as discussed herein that were not previously treatable with fluticasone, e.g., because such formulations could not easily be delivered to a subject. Oral, nasal, and injected formulations generally do not display effective delivery of fluticasone over prolonged periods of time, due to the short half-life of fluticasone in the bloodstream. In contrast, as discussed herein, various aspects of the present invention allow for the improved effectiveness of fluticasone, for instance, over prolonged periods of time.

In one set of embodiments, the composition comprises an ionic salt at a concentration of at least about 5 wt %, and fluticasone and/or a salt, pro-drug, or derivative thereof. The composition may also comprise a nitric oxide donor. The composition comprises, in another set of embodiments, an ionic salt at a concentration of at least about 5 wt %, and a glucocorticoid and/or salt, pro-drug, or derivative thereof. The composition may also comprise a nitric oxide donor. In still another set of embodiments, at least about 80% by weight of the composition comprises an ionic salt at a concentration of at least about 5 wt %, and glucocorticoid and/or salt, pro-drug, or derivative thereof. In some cases, the composition may also comprise a nitric oxide donor.

The composition, in yet another set of embodiments, comprises or consists essentially of water, an ionic salt at a concentration of at least about 5 wt %, potassium chloride, propylene glycol, xanthan gum, glyceryl stearate, cetyl alcohol, a polysorbate, isopropyl myristate, oleic acid, squalane, and/or a glucocorticoid (e.g., fluticasone) and/or a salt, pro-drug, or derivative thereof. The composition, in still another set of embodiments, comprises or consists essentially of water, an ionic salt at a concentration of at least about 5 wt %, potassium chloride, a nitric oxide donor, propylene glycol, xanthan gum, glyceryl stearate, cetyl alcohol, a polysorbate, isopropyl myristate, oleic acid, squalane, and/or a glucocorticoid (e.g., fluticasone) and/or a salt, pro-drug, or derivative thereof.

Another set of embodiments is directed to a composition each of the following compounds at concentrations of no more than +/−20% of the stated concentrations: water at a concentration of about 40% to about 45% by weight, an ionic salt at a concentration of at least about 5% by weight, potassium chloride at a concentration of about 4% to about 6% by weight, a nitric oxide donor at a concentration of about 0% to about 10% by weight, propylene glycol at a concentration of about 5% to about 10% by weight, xanthan gum at a concentration of less than about 1% by weight, glyceryl stearate at a concentration of about 5% to about 10% by weight, cetyl alcohol at a concentration of about 5% to about 10% by weight, a polysorbate surfactant at a concentration of about 1% to about 3% by weight, isopropyl myristate at a concentration of less than about 2% by weight, oleic acid at a concentration of less than about 2% by weight, squalane at a concentration of about 3% to about 5% by weight, and/or a glucocorticoid and/or a salt, pro-drug, or derivative thereof at a concentration of about 0.025% to about 5% by weight.

In another aspect, the present invention is generally directed to a method of treating a subject having or at risk of arthritis. In one set of embodiments, the method comprises applying a cream comprising fluticasone and/or a salt, pro-drug, or derivative thereof to the subject no more than once every 1, 2, or 3 days. In another set of embodiments, the method comprises applying a cream comprising a glucocorticoid and/or salt, pro-drug, or derivative thereof to the subject no more than once every 1, 2, or 3 days.

The method, in yet another aspect, is a method of treating a subject having or at risk of inflammation. According to one set of embodiments, the method includes applying a cream comprising fluticasone and/or a salt, pro-drug, or derivative thereof to the subject no more than once every 1, 2, or 3 days. In another set of embodiments, the method includes acts of applying a cream comprising a glucocorticoid and/or salt, pro-drug, or derivative thereof to the subject no more than once every 1, 2, or 3 days.

Several methods are disclosed herein of administering a subject with a composition for the prevention or treatment of a particular condition. It is to be understood that in each such aspect of the invention, the invention specifically includes, also, the composition for use in the treatment or prevention of that particular condition, as well as use of the composition for the manufacture of a medicament for the treatment or prevention of that particular condition.

Other advantages and novel features of the present invention will become apparent from the following detailed description of various non-limiting embodiments of the invention. In cases where the present specification and a document incorporated by reference include conflicting and/or inconsistent disclosure, the present specification shall control in the absence of clear error. If two or more documents incorporated by reference include conflicting and/or inconsistent disclosure with respect to each other, then the document having the later effective date shall control.

### DETAILED DESCRIPTION

The present invention generally relates to the transdermal delivery of fluticasone and other glucocorticoids. In some aspects, transdermal delivery may be facilitated by the use of a hostile biophysical environment, for example, a high ionic strength environment. One set of embodiments provides a composition for transdermal delivery comprising fluticasone and/or a salt, pro-drug, or derivative thereof, and optionally, a nitric oxide donor. Other glucocorticoids can also be used in some cases. Such compositions may be used to facilitate the delivery of effective amounts of fluticasone or other glucocorticoids systemically or to deeper tissues, rather than only locally or superficially, and in some cases, the effects of fluticasone or other glucocorticoids may be prolonged for unexpectedly long periods of time, e.g., days or weeks. In certain embodiments, the effect of the glucocorticoid lasts at least 1, 2, 3, 4, 5, 6, or 7 days. In certain embodiments, the effect of the glucocorticoid lasts at least 1, 2, 3, or 4 weeks.

While others have suggested delivering fluticasone topically in creams, such formulations have generally been limited to the treatment of skin or surface conditions, such as allergic reactions, eczema, psoriasis, insect bites, atopic dermatitis, contact dermatitis, poison ivy, seborrhea, and the like. However, fluticasone cannot typically be delivered to lower layers of tissue, and/or systemically, e.g., to the bloodstream, using such topical formulations. Accordingly, topical fluticasone has not generally previously been suggested for treating internal inflammation conditions, instead of skin or superficial conditions, such as arthritis, osteoarthritis, rheumatoid arthritis, gout, atherosclerosis, psoriatic arthritis, etc. As discussed herein, when certain types of glucocorticoids such as fluticasone are contained within transdermal delivery vehicles, such as creams, containing hostile biophysical environments (e.g., having high ionic strength, etc., as is discussed herein) and/or a nitric oxide donor, the conditions within the transdermal delivery vehicle may be such that the glucocorticoid is delivered into the skin and in some cases, to an extent that it can reach systemic circulation, and/or target tissues that are below the skin. Under such conditions, the glucocorticoid that is transdermally delivered may be able to enter the bloodstream and/or local tissues directly to target tissues, and thereby treat internal inflammatory condition such as arthritis and the like.

In some cases, formulations described herein that promote transdermal delivery of fluticasone can provide effective relief for longer than 24 hours. Thus, the fluticasone or other glucocorticoid may be delivered over unexpectedly long periods of time, e.g., over several days or even weeks. This is a surprising length of time given that fluticasone normally has an off-rate from the glucocorticoid receptor of about 20-24 hours, and an even shorter half-life in the bloodstream. In some embodiments, after applying a composition as discussed herein to the skin of a subject, the effects of the composition may last for at least two, three, four, five, six, or seven days, or at least one, two, three, or four weeks in some cases. Thus, in some embodiments, a formulation described herein can be effective even when applied less that once in a day, for example once every 2, 3, 4, 5, or 6 days, once per week, or once every second or third weeks, or even less frequently. However, in other embodiments, the formulation can be delivered more often. For example, in some embodiments, the formulation may be delivered once per day, twice per day, three times per day, four or more times per day, etc.

Without wishing to be bound by any theory, it is believed that at least some of the fluticasone (or other glucocorticoid) that is transdermally delivered (e.g., across the skin into the bloodstream), as discussed herein, may become lodged within the skin or elsewhere (e.g., as a depot in lipids within the skin and/or elsewhere within the body), prior to reaching the bloodstream, such that it gradually enters the bloodstream over an extended period of time, allowing the fluticasone to affect the subject over an extended period of time. The fluticasone or other glucocorticoid may thus be lodged within the skin (or just below the skin) and/or within lipids as a “reservoir,” which is slowly released therefrom oven the course of days or weeks into the bloodstream. In addition, in some embodiments, fluticasone or other glucocorticoid may reach the target tissue directly, (e.g., through the skin), without necessarily travelling through the bloodstream. Examples of target tissues include skin, muscle, bone, joints, etc. Other targets for fluticasone or fluticasone or other glucocorticoids include, but are not limited to, receptors on inflammatory cells (e.g., white blood cells, T-cells) in the bloodstream, lymph nodes, or in other target tissues. Thus, for example, the fluticasone or other glucocorticoid can be applied at or near a site of treatment (e.g., to the spine, an ankle, or a knee), for direct delivery (e.g., to tissues internally positioned within the ankle or knee, e.g., to muscle, bone, etc.). In contrast, techniques such as nasal delivery or direct injection result in all of the fluticasone entering the bloodstream at essentially the same time, and during oral delivery, most of the fluticasone is metabolized or inactivated in the liver (the first-pass effect) prior to entering the bloodstream. In addition, rapidly metabolized gluticorticoids such as fluticasone may be advantageous since they are rapidly cleared by the liver to form inactive metabolites which are free of side effects typical of natural gluticorticoids. Thus, in some embodiments, the use of rapidly metabolized glucocorticoids is especially advantageous for safety reasons. However, it should be understood that in certain embodiments, a natural gluticorticoid can be used, e.g., as discussed herein.

Accordingly, in some aspects, the invention provides compositions for the transdermal delivery of pharmaceutical agents such as fluticasone or other glucocorticoids, and in some embodiments, over longer periods of time. These may be applied to the skin of a subject, e.g. a human, to aid in treatment of medical conditions or diseases, and/or the symptoms associated and/or signs thereof. In some embodiments, the invention provides for the treatment of medical conditions or diseases and/or ailments using fluticasone or other glucocorticoids (for example, to treat a subject diagnosed with a medical condition or disease, as described herein), and in some cases, the invention provides for the delivery of a minimum amount of fluticasone or other glucocorticoid to provide effective levels of treatment while limiting side effects. In some cases, the effective dosage of the fluticasone or other glucocorticoid may be lower than the effective dosage of the fluticasone or other glucocorticoid when taken orally, as noted above.

A glucocorticoid is a class of steroid hormones that bind to the glucocorticoid receptor. Glucocorticoids are believed to exert their effects by binding to the glucocorticoid receptor (GR). The activated GR complex may up-regulate the expression of anti-inflammatory proteins in the nucleus (a process known as transactivation) and/or repress the expression of pro-inflammatory proteins in the cytosol by preventing the translocation of other transcription factors from the cytosol into the nucleus (transrepression). Thus, in general, glucocorticoids are believed to be part of the feedback mechanism in the immune system that decreases immune activity or inflammation. Glucocorticoids thus can be used to treat diseases or conditions caused by an overactive immune system, internally, topically, or transdermally, such as allergies, asthma, autoimmune diseases, inflammatory diseases, autoinflammatory diseases, sepsis, eczema, psoriasis, arthritis, insect bites, atopic dermatitis, contact dermatitis, poison ivy, seborrhea, and the like. In addition, in some cases, a composition as is described herein can be used to treat pain, swelling, discomfort, and/or other symptoms caused by inflammation associated with a disease or condition in a joint, muscle or other target tissue. In some cases, the pain, swelling, discomfort, and/or other symptoms may be positioned within deeper tissues, rather than only locally or superficially.

Thus, in some embodiments, various aspects of the invention relate to compositions for delivering a glucocorticoid (such as fluticasone) or a salt, pro-drug, or derivative thereof to a subject. Such compositions can be used to treat inflammatory conditions in target tissue areas below the skin surface in some embodiments. In some embodiments, the composition comprises a glucocorticoid or a salt, pro-drug, or derivative thereof in a hostile biophysical environment for transdermal delivery to the skin of a subject. In some cases, the composition also comprises a nitric oxide donor. In certain embodiments, the composition further comprises one or more compounds that stabilize and/or otherwise promote the efficacy of storage and/or delivery (e.g., with or without a nitric oxide donor).

The glucocorticoid may be naturally-occurring, or artificially synthesized. In one set of embodiments, the glucocorticoid may be a halogenated glucocorticoid, a fluorinated glucocorticoid, or a glucocorticoid having a thioester sidechain at C17. Non-limiting examples of glucocorticoids that may be suitable for use as an anti-inflammatory include, but are not limited to, beclomethasone (pKa of 13-16), budesonide (pKa of 13-15), fluticasone (pKa of 12-14), mometasone (pKa of 12-13), and ciclesonide (pKa of 14-16). The structures of these compounds are respectively shown below:

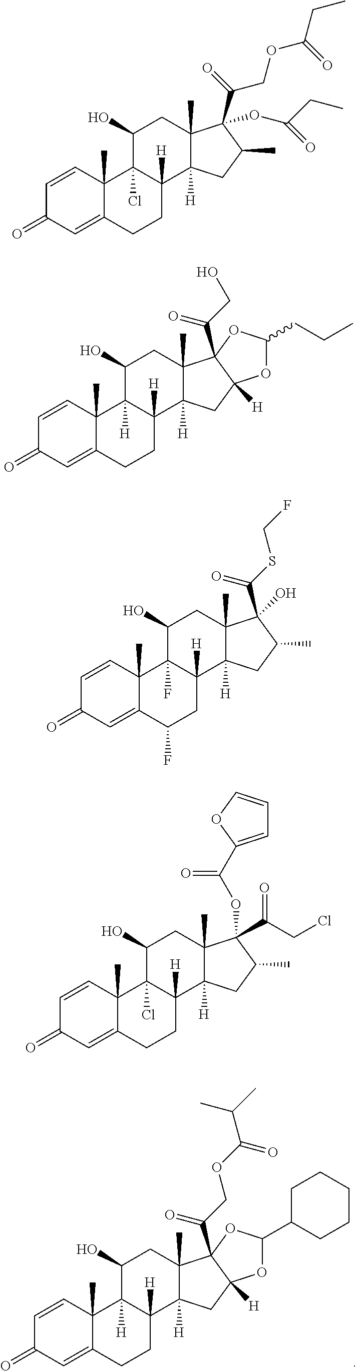

It should be appreciated that depending on the pKa of the glucocorticoid and/or the pH of the composition, the ionized form may be anionic or cationic (e.g., due to protonation). In some cases, the pH of the formulation may be between about 3 and about 7, between about 3 and about 6, between about 3 and about 5, between about 4 and 8, between about 5 and about 8, between about 5 and 8.5, between about 7 and about 11, between about 8 and about 11, between about 9 and about 11, etc. Other pHs are described herein.

In addition, in some cases, the glucocorticoid may an ester, for example, an ester of one of the above compounds (where OR replaces one or more of the OH groups), or other compounds as discussed herein. Examples of such structures include, but are not limited to:

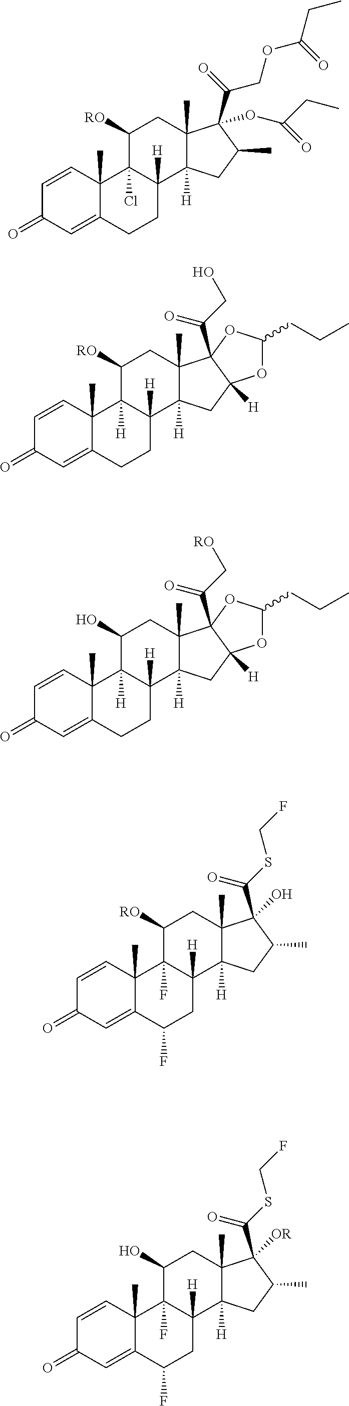

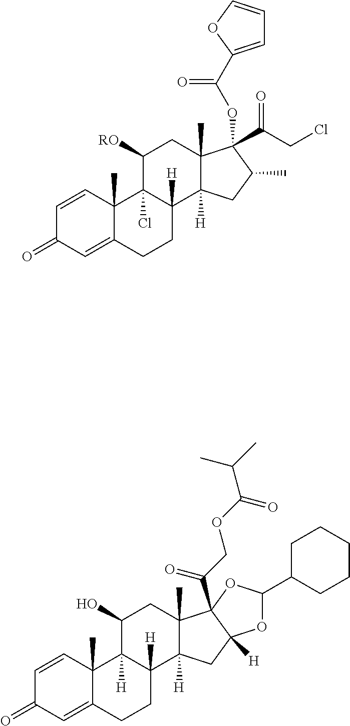

It should be noted that R in the above structures may be any suitable group, e.g., an alkyl group, which may be substituted or unsubstituted, and/or saturated (e.g., an alkenyl) or unsaturated. As non-limiting examples, the ester may be a propionate (where R is C₂H₅—CO—) or a furoate.

Accordingly, various aspects of the invention are directed to compositions comprising glucocorticoids such as fluticasone, or salts or derivatives thereof for transdermal delivery or topical application to a subject. It should be understood that in any embodiment described herein using fluticasone, this is by way of example only, and other embodiments of the invention are directed to other glucocorticoids, salts of glucocorticoids, or derivatives of glucocorticoids, etc., instead of and/or in addition to fluticasone. The glucocorticoid may be any glucocorticoid described herein.

Glucocorticoids such as fluticasone or other pharmaceutical agents (e.g., salts or derivatives of these) may be present at any suitable concentration. For instance, in some cases, the glucocorticoid may be present at a concentration of at least about 0.01%, at least about 0.025%, at least about 0.05%, at least about 0.75%, at least about 0.1%, at least about 0.3%, at least about 0.5%, at least about 0.7%, at least about 1%, at least about 2%, at least about 3%, at least about 4%, at least about 5%, at least about 6%, at least about 7%, at least about 7.5%, at least about 8%, at least about 9%, or at least about 10% by weight of the composition. In certain embodiments, the glucocorticoid may be present at a concentration of no more than about 0.025%, no more than about 0.05%, no more than about 0.075%, no more than about 1%, no more than about 2%, no more than about 3%, no more than about 4%, no more than about 5%, no more than about 6%, no more than about 7%, no more than about 8%, no more than about 9%, no more than about 10%, no more than about 12%, no more than about 15%, or no more than about 20% by weight of the composition. Combinations of any of these are also possible in certain embodiments. In addition, the glucocorticoid may be present in native form and/or as one or more salts, pro-drugs, or derivatives. For example, if a glucocorticoid such as fluticasone is present, it may be used in its native form, and/or as one or more salts, pro-drugs, or derivatives, e.g., the furoate salt, the propionate salt, the sodium salt, the potassium salt, the magnesium salt, the lysine salt, the arginine salt, the lactate salt, the citrate salt, etc. Specific non-limiting examples of forms of glucocorticoids useful in the present invention include fluticasone propionate, fluticasone furoate, beclomethasone dipropionate, mometasone furoate, etc. For various forms of the pharmaceutical agent, “by weight of the composition” includes the entire form of the pharmaceutical agent, e.g., for a salt form of the agent itself as well as any counterion, etc. The amount of the pharmaceutical agent may be determined in a composition, for example, using techniques such as HPLC or HPLC/MS that are known to those of ordinary skill in the art.

Many glucocorticoids are readily commercially available. For example, beclomethasone, budesonide, fluticasone, mometasone and ciclesonide are all sold commercially. In addition, in some cases, the glucocorticoid may be obtained as a racemic mixture. However, in other cases, one of the enantiomers, diastereomers, or other stereoisomers may be present in an amount greater than the other. For example, at least about 60%, at least about 70%, at least about 80%, at least about 90%, or at least about 95% of the glucocorticoid within the composition may be present as one of the isomers.

The composition may also comprise a nitric oxide donor in some embodiments, for example, L-arginine and/or L-arginine hydrochloride. In some cases, such a nitric oxide donor may be used to increase localized blood flow at the site where the composition is applied, which may enhance delivery of the glucocorticoid. The nitric oxide donor may be present at any suitable concentration within the composition. For instance, in some cases, the nitric oxide donor is present at a concentration of at least about 1%, at least about 2%, at least about 3%, at least about 4%, at least about 5%, at least about 6%, at least about 7%, at least about 7.5%, at least about 8%, at least about 9%, or at least about 10% by weight of the composition. In some cases, one or more nitric oxide donors (e.g., 2, 3, 4, 5, 6, 7, 8, 9, 10, or more nitric oxide donors) may be used. In some cases, there may be no more than 3, 5, 7, or 10 nitric oxide donors present within the composition.

A “nitric oxide donor,” as used herein, is a compound that is able to release nitric oxide and/or chemically transfer the nitric oxide moiety to another molecule, directly or indirectly, for example, through a biological process. The nitric oxide donor may release nitric oxide into the skin, and/or tissues such as muscles and/or elements of the circulatory system in close proximity to the surface of the skin. Non-limiting examples of nitric oxide donors include arginine (e.g., L-arginine and/or D-arginine), arginine derivatives (e.g., L-arginine hydrochloride and/or D-arginine hydrochloride), nitroglycerin, polysaccharide-bound nitric oxide-nucleophile adducts, N-nitroso-N-substituted hydroxylamines, 1,3-(nitrooxymethyl)phenyl-2-hydroxybenzoate, etc., and/or any combination of these and/or other compounds.

Besides L-arginine and L-arginine hydrochloride, other non-limiting examples of nitric oxide donors include D,L-arginine, D-arginine, or alkyl (e.g., ethyl, methyl, propyl, isopropyl, butyl, isobutyl, tert-butyl, etc.) esters of L-arginine and/or D-arginine (e.g., a methyl ester, an ethyl ester, a propyl ester, a butyl ester, etc.) and/or salts thereof, as well as other derivatives of arginine and other nitric oxide donors. For instance, non-limiting examples of pharmaceutically acceptable salts include hydrochloride, glutamate, butyrate, or glycolate (e.g., resulting in L-arginine glutamate, L-arginine butyrate, L-arginine glycolate, D-arginine hydrochloride, D-arginine glutamate, etc.). Still other examples of nitric oxide donors include L-arginine-based compounds such as, but not limited to, L-homoarginine, N-hydroxy-L-arginine, nitrosylated L-arginine, nitrosylated L-arginine, nitrosylated N-hydroxy-L-arginine, nitrosylated N-hydroxy-L-arginine, citrulline, ornithine, linsidomine, nipride, glutamine, etc., and salts thereof (e.g., hydrochloride, glutamate, butyrate, glycolate, etc.), and/or any combination of these and/or other compounds. Still other non-limiting examples of nitric oxide donors include S-nitrosothiols, nitrites, 2-hydroxy-2-nitrosohydrazines, or substrates of various forms of nitric oxide synthase. In some cases, the nitric oxide donor may be a compound that stimulates endogenous production of nitric oxide in vivo. Examples of such compounds include, but are not limited to, L-arginine, substrates of various forms of nitric oxide synthase, certain cytokines, adenosine, bradykinin, calreticulin, bisacodyl, phenolphthalein, OH-arginine, or endothelein, and/or any combination of these and/or other compounds.

Accordingly, it should be understood that, in any of the embodiments described herein that describe L-arginine and/or L-arginine hydrochloride, other nitric oxide donors may also be used instead, or in combination with, L-arginine and/or L-arginine hydrochloride, in other embodiments of the invention.

In some cases, the concentration of the nitric oxide donor within the composition may be tailored to have a duration of effective treatment of at least about 3 hours, at least about 5 hours, or at least about 8 hours or more in certain instances. The duration may also be controlled, for instance, by controlling the concentration of a penetrating agent used in conjunction with the nitric oxide donor. Penetration agents are discussed in more detail herein. The actual concentration for a particular application can be determined by those of ordinary skill in the art using no more than routine experimentation, for example, by measuring the amount of transport of the nitric oxide donor as a function of concentration in vitro across cadaver skin or suitable animal models, skin grafts, synthetic model membranes, human models, or the like.

As a particular non-limiting example, in certain embodiments, nitric oxide is provided using L-arginine, for example, at a concentration of at least about 0.5% by weight (wt % or w/v) of L-arginine (optionally with one or more penetrating agents as discussed herein, for example, a penetrating agent able to create a hostile biophysical environment), at least about 0.75 wt %, at least about 1 wt %, at least about 2 wt %, at least about 3 wt %, at least about 5 wt %, at least about 7 wt %, at least about 10 wt %, or at least about 15 wt %. The L-arginine may be present in a suitable delivery vehicle, such as a cream or a lotion. L-arginine may be particularly useful in some cases due to its low toxicity, its high solubility, and/or its low cost. In some cases, L-arginine HCl may be used in addition to or instead of L-arginine. Other examples of nitric oxide donors are discussed in International Patent Application No. PCT/US2005/005726, filed Feb. 23, 2005, entitled “Topical Delivery of a Nitric Oxide Donor to Improve Body and Skin Appearance,” by E. T. Fossel, published as WO 2005/081964 on Sep. 9, 2005, incorporated herein by reference.

The composition may also comprise a hostile biophysical environment to fluticasone or other glucocorticoids in some embodiments. In a hostile biophysical environment, the environment surrounding the pharmaceutical agent (e.g., a fluticasone or other glucocorticoids, etc.) may be such that the pharmaceutical agent is in a chemically and/or energetically unfavorable environment, relative to the skin (e.g., the chemical potential and/or the free energy of the pharmaceutical agent within the hostile biophysical environment is significantly greater than the chemical potential and/or the free energy of the pharmaceutical agent within the skin, thus energetically favoring transport into the skin), especially the stratum corneum.

A hostile biophysical environment of the invention can comprise, in various embodiments, high ionic strength, a high concentration of osmotic agents such as ureas, sugars, or carbohydrates, a high pH environment (e.g., greater than about 7, greater than about 8, greater than about 9, greater than about 10, greater than about 11, greater than about 12, or greater than about 13), a low pH environment (less than about 5, less than about 4, less than about 3 or less than about 2), highly hydrophobic components, or highly hydrophilic components or other substances that cause an increase in the chemical potential and/or free energy of the pharmaceutical agent, or any combination of two or more of these and/or other compounds. A hydrophobic component may, in some embodiments, have an octanol-water partition coefficient of at least about 100, at least about 1000, at least about 10⁴, at least about 10⁵, or more in some cases. Similarly, a hydrophilic component may have an octanol-water partition coefficient of less than about 0.01, less than about 10⁻³, less than about 10⁻⁴, or less than about 10⁻⁵ in some cases.

In some cases, the composition defines the biophysical hostile environment. In certain instances, a pharmaceutical agent may be packaged in such a way that it is carried into tissue and/or its charge is neutralized by derivitization and/or by forming a neutral salt. Examples of biophysically hostile environments include, but are not limited to, high ionic strength environments (e.g., by the addition of ureas, sugars, carbohydrates, and/or ionic salts such as lithium chloride, sodium chloride, potassium chloride, calcium chloride, magnesium chloride, choline chloride, sodium fluoride, lithium bromide, sodium citrate, etc.), as well as combinations of these and/or other agents, for instance at high ionic strengths (for example, greater than about 0.25 M, greater than about 1 M, greater than about 2 M, greater than about 3 M, greater than about 5 M, greater than about 10 M, greater than about 15 M, greater than about 20 M, greater than about 25 M, etc., or in some cases, between about 0.25 M and about 15 M, between about 1 M and about 15 M, between about 5 M and about 15 M, between about 10 M and about 15 M, etc.); high or low pH environments (e.g., by adding pharmaceutically acceptable acids or bases, for example, such that the pH is between about 3 and about 7, between about 3 and about 6, between about 3 and about 5, between about 4 and 8, between about 5 and about 8, between about 5 and 8.5, between about 7 and about 11, between about 8 and about 11, between about 9 and about 11, etc.); or highly hydrophobic environments (e.g., by decreasing water content and increasing lipid, oil and/or wax content of the environment). In some embodiments, the ionic strength is any amount greater than two times the physiological ionic strength of blood. The ionic strength of a composition can be readily controlled in certain embodiments by controlling the amounts or concentrations of one or more of the salts present in the composition, e.g., by controlling the amount of sodium chloride, magnesium chloride, choline chloride, sodium citrate, etc., and/or other salts.

Other highly charged molecules such as polylysine, polyglutamine, polyaspartate, etc., or copolymers of such highly charged amino acids may also be used in certain embodiments to create the hostile biophysical environment. Non-limiting examples of delivery vehicles which would be carried into tissue includes liposomes or emulsions of collagen, collagen peptides or other components of skin or basement membrane. Non-limiting examples of neutralization of charge include delivery of the pharmaceutical agent in the form or an ester or salt which is electronically neutral. In some embodiments, the hostile biophysical environment may include any two or more of these conditions. For instance, the hostile biophysical environment may include high ionic strength and a high pH or a low pH, a highly hydrophobic environment and a high pH or a low pH, a highly hydrophobic environment that includes liposomes, or the like.

A hostile biophysical environment may also be created in some embodiments by placing a pharmaceutical agent that is relatively highly charged into a hydrophobic, oily environment such as in an oil-based cream or lotion containing little or no water. Absorption may further be aided by combining the use of hostile biophysical environments with the use of penetrating agents, as further described herein.

Additional examples of such compositions are discussed in International Patent Application No. PCT/US2005/013228, filed Apr. 19, 2005, entitled “Transdermal Delivery of Beneficial Substances Effected by a Hostile Biophysical Environment,” by E. Fossel, published as WO 2005/102282 on Nov. 3, 2005, incorporated herein by reference. Accordingly, certain embodiments of the invention are generally directed to compositions for transdermal delivery to the skin of a subject comprising a nitric oxide donor, a hostile biophysical environment, and a glucocorticoid such as fluticasone, a salt, pro-drug, or a derivative thereof, or the like.

Thus, according to some embodiments of the invention, compositions comprising a relatively high salt composition (e.g., high chloride content) may be effective for transdermal delivery of a glucocorticoid such as fluticasone (including salts, pro-drugs, or derivatives thereof). In some embodiments, a salt-enhanced delivery (e.g., in a composition having at least 2% salt, at least 5% salt, at least 10% salt, at least 15% salt, or higher as described herein) is particularly effective when the pH of the composition is optimized to ionize the compound being delivered (e.g., at least about 80%, at least about 90%, at least about 95%, or about 99% or more) is ionized. It should be appreciated that depending on the pKa of the compound and the pH of the composition, the ionized form may be anionic or cationic (e.g., due to protonation). In some embodiments, a compound may contain several ionizable groups each having a different pKa. In some embodiments, it is sufficient for at least 1, 2, or 3 of the groups to be ionized for the salt-enhanced delivery to be effective. In some embodiments, an ionizable group is sufficiently ionized if the pH of the composition is at least 1 pH unit, or at least 2 pH units (e.g., 1, 1-2, 2-3, or more pH units) below the pKa of the group and it is cationic (due to protonation) below its pKa. Similarly, in some embodiments, an ionizable group is sufficiently ionized if the pH of the composition is at least 1 pH unit, or at least 2 pH units (e.g., 1, 1-2, 2-3, or more pH units) above the pKa of the group and it is anionic (due to deprotonation) above its pKa. In some embodiments, the presence of magnesium chloride, for example at 0.1-5% by weight, can help stabilize compositions containing compounds with relatively high pKas (e.g., above 8.0, above 9.0, above 10.0 or higher). In some embodiments, the pH of a composition may be maintained using a buffer. However, the pH of compositions of the invention may also stable without a buffer. In some embodiments, a desired pH can be established by titrating the mixture with an acid (e.g., HCl) or a base (e.g., NaOH). The pH of the resulting composition (e.g., when formulated as an emulsion as described herein) can be stable (e.g., sufficiently for the composition to be effective for transdermal delivery) for extended periods of time (e.g., weeks, months, or 1 or more years).

According to some aspects of the invention, a relatively high salt concentration, for example at least about 2% (e.g., about 5%, about 10% about 15%, about 20% about 25%, about 25-50%, weight percent) is useful to provide a hostile biophysical environment that promotes transdermal delivery of glucocorticoids such as fluticasone. In some embodiments, emulsions described herein, for example, containing a stabilization polymer and/or a polysorbate surfactant and/or propylene glycol (or a low molecular weight glycol, or a polyglycol such as polyethylene glycol or other polyglycol can be used; however, it should be appreciated that glycols with an even number of carbons can be toxic, particularly for smaller glycols such as ethylene glycol and butylene glycol, and should be avoided or excluded in some cases) are unexpectedly effective at stabilizing the glucocorticoid in the high salt composition in a form that remains effective for an extended period, for example, retaining rapid transdermal delivery of the glucocorticoid for at least several weeks or months.

According to certain aspects of the invention, a high salt composition containing a glucocorticoid such as fluticasone may be stable when formulated as an emulsion (e.g., a water in oil emulsion or an oil in water emulsion, for example, including one or more of a stabilization polymer and/or a polysorbate surfactant and/or propylene glycol (or other low molecular weight glycol, or a polyglycol) as described herein). In some embodiments, the pH of the composition comprising the emulsion and high salt concentration is selected to ionize the compound being delivered as described herein. Thus, in one set of embodiments, the composition may be present as an emulsion. As known by those of ordinary skill in the art, an emulsion typically includes a first phase (e.g., a discontinuous phase) contained within a second fluid phase (e.g., a continuous phase). The pharmaceutical agent (e.g., a glucocorticoid such as fluticasone) may be present in either or both phases. In addition, other materials such as those described herein may be present in the same phase as the pharmaceutical agent. In some embodiments, an emulsion may be prepared to contain a drug (or other pharmaceutical agent) of interest in a hostile biophysical environment, and optionally one or more of a stabilization polymer, propylene glycol, and/or a polysorbate surfactant. An emulsion may also comprise a nitric oxide donor in some embodiments, for example, L-arginine and/or L-arginine hydrochloride.

In some embodiments, an emulsion is prepared by mixing a first aqueous preparation (e.g., a water phase) with a second non-aqueous preparation (e.g., an oil or lipid phase). Drugs or other pharmaceutical agents that are water-soluble may be added to the first aqueous preparation (e.g., prior to mixing with the second non-aqueous preparation). Drugs or other pharmaceutical agents that are water insoluble (or relatively water insoluble) may be added to the second non-aqueous preparation (e.g., prior to mixing with the first aqueous preparation). Drugs or other pharmaceutical agents that are partially water soluble may be added to one phase, or may be split between the two phases prior to mixing. The split between the two phases will depend on the amount of drug (or other pharmaceutical agent) that is being added, the composition (e.g., the nature and the amount of other chemicals or agents) of the first and second preparations, the pH, the temperature, other physical or chemical factors, and/or a combination thereof. For example, if a drug of interest is soluble at a 1% level in the aqueous (e.g., water or buffer) phase, but a 2% level of the drug is required in the emulsion, then the drug may also be added to the non-aqueous (e.g., lipid) phase at a 1% level. In some embodiments, a drug that is less than 1% soluble in an aqueous phase is provided in the non-aqueous phase prior to mixing. However, it should be appreciated that other percentages and/or splits between the two phases may be used.

In some embodiments, the pH of one or both of the first and second preparations is adjusted to optimize the solubility of the drug being used. In some embodiments, a high salt concentration is used. In order to prevent a high salt concentration from breaking down an emulsion, one or more emulsifying agents may be used in some cases. In some embodiments, the mixing time may be adjusted to promote appropriate mixing and/or emulsion formation.

In some embodiments, the temperature of the first and/or second preparation may be controlled to promote solubility, mixing, and/or emulsion formation. In some embodiments, the temperature of one or both preparations and/or of the mixing may be set at 25° C. or higher (e.g., 30° C. or higher, 40° C. or higher, 50° C. or higher, 60° C. or higher, 70° C. or higher, or 80° C. or higher). For example, the temperature may be at between 30° C. and 90° C., between 40° C. and 80° C., at around 50° C., at around 60° C., or at around 70° C.

Emulsions of the invention may be packaged using any suitable format (e.g., in a tube, a pump-actuated container, or any other suitable form), in certain embodiments of the invention. For example, in some embodiments, an emulsion may be added to a surface of a patch or bandage. The emulsion may also be applied to the skin of a subject as a cream, gel, liquid, lotion, spray, aerosol, or the like.

Accordingly, in some embodiments, various aspects of the invention relate to methods and compositions for preparing and/or manufacturing drug formulations for transdermal delivery. In one set of embodiments, the present invention is generally directed to emulsions that contain one or more drugs or other pharmaceutical agents described herein for transdermal application. In some embodiments, certain aspects of the invention are useful for preparing emulsions that contain one or more drugs (or other pharmaceutical agents) in a hostile biophysical environment. In some embodiments, the hostile biophysical environment is a high salt concentration (e.g., a high concentration of one or more salts), for example, as described herein.

It should be appreciated that methods and compositions of the invention may be used with any suitable drug or pharmaceutical agent. In some embodiments, for example, an oral drug may be formulated for transdermal delivery using one or more compositions or methods described herein. A transdermal formulation may be useful to deliver a locally effective amount of a drug (or other pharmaceutical agent) to a subject (e.g., a human) without causing unwanted side effects associated with systemic levels required for effectiveness when the drug is administered orally. Accordingly, a transdermal formulation may be useful to deliver an amount of a drug that is sufficient to cause a desired effect (e.g., a therapeutic effect) but that is lower than the total amount of the drug that would be administered to a subject (e.g., a human) if it were provided orally.

Thus, another aspect of the invention provides for the delivery of pharmaceutical agents (e.g., drugs, biological compounds, etc.) into the body, and such treatments may be systemic or localized, e.g., directed to a specific location of the body of a subject, such as the head, one or more specific muscles, an arm, a leg, etc., depending on the specific application. In some embodiments, compositions described herein are applied to a skin region near a target tissue site, for example on a knee, elbow, wrist, ankle, or other joint. However, in some cases, the composition need not be applied to a skin region at or near a target tissue site, for example, in cases where the systemic circulation is adequate to deliver fluticasone or other glucocorticoid to the target tissue.

In some cases, the subject may be one that has or is at risk of a skin condition, such as allergic reactions, eczema, psoriasis, insect bites, atopic dermatitis, contact dermatitis, poison ivy, or seborrhea. In certain instances, the subject may be one that has or is at risk for an internal inflammation condition or a systemic inflammatory disease or condition, such as arthritis, osteoarthritis, rheumatoid arthritis, gout, atherosclerosis, allergies, asthma, autoimmune diseases, autoinflammatory diseases, sepsis, etc. Additional non-limiting examples include rheumatoid arthritis, arachnoiditis, spondylitis, neuralgia, fibromyalgia, injuries, post-surgical inflammation, or the like.

In addition, in some embodiments, various aspects of the invention relate to methods and formulations for delivering a compound locally at a fraction of the systemic dose required using oral delivery. In some embodiments, a hostile biophysical environment may be evaluated for enhancing local delivery through topical application. Depending on the therapeutic application, an appropriate delivery configuration (e.g., a combination of compound concentration, hostile biophysical environment, cream, patch, etc.) can be used to reduce the systemic amount of the compound required for an effective therapeutic application.

In some aspects of the invention, a composition of the invention is administered to a subject using a delivery vehicle such as a cream, gel, liquid, lotion, spray, aerosol, or transdermal patch. Methods and compositions such as any of those discussed herein may also be used to prepare a composition that is sterile or that has a low microbial content, in some embodiments. In one set of embodiments, a composition of the invention may be applied or impregnated in a bandage or a patch applied to the skin of a subject. In some embodiments, a patch has a skin contacting portion made of any suitable material that is covered or impregnated with a cream or emulsion described herein, wherein the skin contacting portion may be supported by a backing, one or both of which may have an adhesive segment or other configuration for attaching to the skin surface of a subject.

A “subject,” as used herein, means a human or non-human animal. Examples of subjects include, but are not limited to, a mammal such as a dog, a cat, a horse, a donkey, a rabbit, a cow, a pig, a sheep, a goat, a rat (e.g., Rattus norvegicus), a mouse (e.g., Mus musculus), a guinea pig, a hamster, a primate (e.g., a monkey, a chimpanzee, a baboon, an ape, a gorilla, etc.), or the like. Such delivery vehicles may be applied to the skin of a subject, such as a human subject. Examples of delivery vehicles are discussed herein.

The delivery vehicle may promote transfer into the skin of an effective concentration of the nitric oxide donor and/or the pharmaceutical agent, directly or indirectly. For instance, the delivery vehicle may include one or more penetrating agents, as further described herein. Those of ordinary skill in the art will know of systems and techniques for incorporating a nitric oxide donor and/or a pharmaceutical agent within delivery vehicles such as a cream, gel, liquid, lotion, spray, aerosol, or transdermal patch. In some cases, the concentration of the nitric oxide donor, and/or a pharmaceutical agent in the delivery vehicle can be reduced with the inclusion of a greater amount or concentration of penetrating agent, or increased to lengthen the beneficial effect. In one set of embodiments, the nitric oxide donor and/or the pharmaceutical agent may be used in conjunction with an adjunct, such as theophylline (for example, at 10% weight by volume). In some embodiments, certain aspects of the invention relate to a patch that comprises a composition of the invention (e.g., with or without a nitric oxide donor, and with or without one or more stabilizing compounds). In some embodiments, a composition is in the form of a cream or ointment that is incorporated into the patch. However, other configurations also may be used.

Other materials may be present within the delivery vehicle, for example, buffers, preservatives, surfactants, etc. For instance, the cream may include one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A and D, triethanolamine, methylparaben, aloe vera extract, imidazolidinyl urea, propylparaben, PND, and/or BHA.

As specific non-limiting examples, a cream may have one or more of (w/v): water (20-80%), white oil (3-18%), glyceryl stearate (0.25-12%), squalene (0.25-12%), cetyl alcohol (0.1-11%), propylene glycol stearate (0.1-11%), wheat germ oil (0.1-6%), polysorbate 60 (0.1-5%), propylene glycol (0.05-5%), collagen (0.05-5%), sorbitan stearate (0.05-5%), vitamin A (0.02-4%), vitamin D (0.02-4%), vitamin E (0.02-4%), triethanolamine (0.01-4%), methylparaben (0.01-4%), aloe vera extract (0.01-4%), imidazolidinyl urea (0.01-4%), propylparaben (0.01-4%), BHA (0.01-4%), L-arginine hydrochloride (0.25-25%), sodium chloride or sodium citrate (0.25-25%), magnesium chloride (0.25-25%), and/or choline chloride (0.25-25%). The percentages of each compound can vary (or the compound may be absent in some cases), for example, 1%, 2%, 3%, 4%, 5%, 6%, 7%, 8%, 9%, 10%, 11%, 12%, 13%, 14%, 15%, 20%, etc.

In another embodiment, the cream may include a pharmaceutical agent, for instance, a glucocorticoid such as fluticasone, e.g., as described herein, and one or more of the following, in any suitable amount: water (e.g., 20-80%), L-arginine hydrochloride (e.g., 0-25%), sodium chloride or sodium citrate (e.g., 0-25%), potassium chloride (e.g., 0-25%), glyeryl steareate (e.g., 0-15%), cetyl alcohol (e.g., 0-15%), squalene (e.g., 0-15%), isopropyl mysterate (e.g., 0-15%), oleic acid (e.g., 0-15%), Tween 20 (e.g., 0-10%), and/or butanediol (e.g., 0-10%). The percentages of each compound can vary (or the compound may be absent in some cases), for example, 1%, 2%, 3%, 4%, 5%, 6%, 7%, 8%, 9%, 10%, 11%, 12%, 13%, 14%, 15%, 20%, etc.

In some embodiments, the cream may include fluticasone or another glucocorticoid, and one or more ionic salts at a concentration at least sufficient to produce a hostile biophysical environment with respect to the pharmaceutical agent. For example, the cream may include one or more of (w/v): a charged and/or hydrogen bonding entity (0.001-30%), choline chloride (1-30%), sodium chloride or sodium citrate (2-30%), and/or magnesium chloride (1-20% w/v). In another example, the cream may include one or more of (w/v): L-arginine hydrochloride (2.5-25%), choline chloride (10-30%), sodium chloride or sodium citrate (5-20%), and/or magnesium chloride (5-20%). In still another example, the cream may include one or more of (w/v): creatine (0.001-30%), inosine (0.001-30%), choline chloride (1-30%), sodium chloride or sodium citrate (2-30%), magnesium chloride (1-20%), L-arginine (0.1-25%), and/or theophylline (0.1-20%). In some cases, the cream may also contain L-arginine hydrochloride (0-12.5% w/v) and/or theophylline (0-10% w/v). The percentages of each compound can vary (or the compound may be absent in some cases), for example, 1%, 2%, 3%, 4%, 5%, 6%, 7%, 8%, 9%, 10%, 11%, 12%, 13%, 14%, 15%, 20%, etc. In these examples, choline chloride, sodium chloride, sodium citrate, and/or magnesium chloride can be used to provide a high ionic strength environment.

In another embodiment, the invention is directed to a composition for transdermal delivery to the skin of a subject, comprising one or more of: water (35-45%), an ionic salt such as sodium chloride or sodium citrate (at least 5%), potassium chloride (4-6%), a nitric oxide donor such as L-arginine or L-arginine HCl (5-10%), propylene glycol (5-10%), xanthan gum (1% or less), glyceryl stearate (5-10%), cetyl alcohol (5-10%), a polysorbate surfactant such as polysorbate 20 (1-3%), isopropyl myristate (2% or less), oleic acid (2% or less), squalane (3-5%), and/or a glucocorticoid and/or a salt, pro-drug, or derivative thereof (5-10%). The percentages of each compound can vary (or the compound may be absent in some cases), for example, 1%, 2%, 3%, 4%, 5%, 6%, 7%, 8%, 9%, 10%, 11%, 12%, 13%, 14%, 15%, 20%, etc.

In addition, in some cases, the percentages may vary from the ones described above, e.g., by +/−5%, +/−10%, +/−15%, +/−20%, etc.

In some embodiments, the composition may include an antioxidant, which may be able to reduce or inhibit the oxidation of other molecules within the composition. Examples of suitable antioxidants include, but are not limited to, glutathione, vitamin C, and vitamin E as well as enzymes such as catalase, superoxide dismutase and various peroxidases. The antioxidant may be present in any suitable concentration. For example, the antioxidant may be present at a concentration of at least about 0.1%, at least about 0.3%, at least about 0.5%, at least about 0.7%, at least about 1%, at least about 2%, at least about 3%, at least about 4%, or at least about 5% by weight of the composition. In certain embodiments, the pharmaceutical agent may be present at a concentration of no more than about 0.2%, no more than about 0.5%, no more than about 1%, no more than about 2%, no more than about 3%, no more than about 4%, or no more than about 5% by weight of the composition.

As mentioned, in some cases, the composition may be allow for surprisingly long periods of delivery of fluticasone or other glucocorticoids to a subject after the initial application. For example, after the composition has been applied to the skin of a subject, the effects of the fluticasone or other glucocorticoid may be felt by the subject for a period of days or weeks, before an additional treatment is needed. For instance, the composition may be applied to a subject at least two, three, four, five, six, or seven days, or at least one, two, three, or four weeks after an earlier treatment to the subject. These are unexpectedly long since fluticasone normally has an off-rate from the glucocorticoid receptor of about 20-24 hours, and an even shorter half-life within the bloodstream.

Another set of embodiments is generally directed to compositions having relatively high temperature stability. For example, the composition may be stable at elevated temperatures such as at least 40° C. (at least about 104° F.) for periods of time of at least about a day. In some embodiments, for instance, a composition of the present invention may further include a stabilization polymer, propylene glycol, and a polysorbate surfactant. Non-limiting examples of stabilization polymers include xanthan gum, KELTROL® BT and/or KELTROL® RD; an example of a polysorbate surfactant is Polysorbate 20. Additional examples are discussed herein.

Such a combination of components to create high temperature stability are surprising, since compositions involving any two of these components (but not the third) were found to lack such high temperature stabilization properties. It is not currently known why this combination of components is remarkably effective at facilitating relatively high temperature stability of the compositions discussed herein, as these components are not known to participate in any significant chemical reactions with each other, and high temperature stability is greatly reduced when one of the components is removed. In addition, propylene glycol is not known to work in pharmaceutical compositions as a stabilizing agent.

For instance, in one set of embodiments, a composition may be determined to be one that has high temperature stability by determining whether the composition exhibits phase separation over a relatively long period of time, e.g., over at least an hour, at least about 2 hours, at least a day, at least about a week, at least about 4 weeks, etc. For example, in some embodiments, a composition is exposed to ambient temperature and pressure for at least 1 hour, and the composition is then analyzed to determine whether the composition exhibits phase separation or a change in phase. A stable compound is one that exhibits no phase separation, whereas an unstable compound may exhibit phase separation. Such stability may be useful, for example, for storage of the composition, transport of the composition, shelf life, or the like.

As used herein, a “stabilization polymer” is a polymer that comprises xanthan gum, a xanthan gum derivative, and/or a xanthan gum equivalent, for example, KELTROL® BT and/or KELTROL® RD, KELZAN® XC, KELZAN® XCD, KELZAN® D, KELZAN® CC, XANTURAL® 180, XANTURAL® 75, or the like, all of which can be obtained commercially from various suppliers. In some embodiments, combinations of these and/or other polymers are also possible. In some cases, the stabilization polymer is chosen to be one which is at least generally regarded as safe for use in humans. In addition, in certain embodiments, the stabilization polymer is produced synthetically, and/or one which has been purified to some degree. The stabilization polymer may have any suitable molecular weight, for example, at least about 1 million, at least about 2 million, at least about 5 million, at least about 10 million, at least about 25 million, or at least about 50 million.

The stabilization polymer may be present at any suitable concentration within the composition. For example, the stabilization polymer may be present at a concentration of at least about 0.1%, at least about 0.2%, at least about 0.3%, at least about 0.4%, at least about 0.5%, at least about 0.6%, at least about 0.7%, at least about 0.8%, at least about 0.9%, or at least about 1% by weight of the composition. In some embodiments, the stabilization polymer may be present at a concentration of no more than about 0.1%, no more than about 0.2%, no more than about 0.4%, no more than about 0.6%, no more than about 0.8%, no more than about 1%, no more than about 2%, no more than about 3%, no more than about 4%, no more than about 5%, no more than about 7%, no more than about 10%, no more than about 12%, no more than about 15%, or no more than about 20% by weight of the composition. In some cases, more than one stabilization polymer may be present, and each stabilization polymer may be present in any suitable amount.

As a specific example, in certain embodiments, the stabilization polymer consists essentially of KELTROL® BT and/or KELTROL® RD. In certain instances, the stabilization polymer may have a fixed ratio of KELTROL® BT and/or KELTROL® RD, for example, 1:1 or 3:5 by weight. In another example, the KELTROL® BT may be present at a concentration of about 0.3% by weight and the KELTROL® RD may be present at a concentration of 0.5% by weight of the composition, or one or both of these may be present at one of the other concentrations described above. Combinations of these and/or other stabilization polymers are also contemplated in other embodiments, e.g., KELTROL® BT and xanthan gum, KELTROL® RD and xanthan gum, etc. In some cases, thickening agents can be used instead of, or in conjunction with a stabilization polymer. Many thickening agents can be obtained commercially. Thickening agents include those used in the food industry, or are GRAS agents (generally regarded as safe), e.g., alginin, guar gum, locust bean gum, collagen, egg white, furcellaran, gelatin, agar, and/or carrageenan, as well as combinations of these and/or other stabilization polymers. It should thus be appreciated that, in the specification herein, references to stabilization polymers, in other embodiments, should be understood to also include thickening agents in conjunction or instead of stabilization polymers,

Propylene glycol can be obtained commercially, and can be present as any stereoisomer or racemic mixture of isomers. It may also be present at any suitable concentration. For instance, propylene glycol may be present at a concentration of at least about 1%, at least about 2%, at least about 3%, at least about 4%, at least about 5%, at least about 6%, at least about 7%, at least about 8%, at least about 9%, or at least about 10% by weight of the composition. In some embodiments, propylene glycol may be present at a concentration of no more than about 2%, no more than about 4%, no more than about 6%, no more than about 8%, no more than about 10%, no more than about 12%, no more than about 15%, no more than about 20%, or no more than about 25% by weight of the composition. In some cases, other glycols can be used in conjunction or instead of propylene glycol, such as butylene glycol. Accordingly, it should thus be appreciated that, in the specification herein, references to propylene glycol, in other embodiments, should be understood to also include other glycols (e.g., a low molecular weight glycol, or a polyglycol, as described herein) in conjunction or instead of propylene glycol.

In addition, a polysorbate surfactant can also be present any suitable concentration within the composition. For instance, in some cases, the polysorbate surfactant may be present at a concentration of at least about 1%, at least about 2%, at least about 3%, at least about 4%, at least about 5%, at least about 6%, at least about 7%, at least about 8%, at least about 9%, or at least about 10% by weight of the composition. In certain embodiments, the polylsorbate surfactant may be present at a concentration of no more than about 2%, no more than about 4%, no more than about 6%, no more than about 8%, no more than about 10%, no more than about 12%, no more than about 15%, no more than about 20%, or no more than about 25% by weight of the composition A “polysorbate surfactant,” as used herein, is a surfactant comprising a polysorbate. For example, the surfactant may comprise sorbitan monolaurate, sorbitan monopalmitate, sorbitan monostearate, sorbitan monooleate, or another sorbitan salt. In some cases, the polysorbate surfactant has a molecular formula:

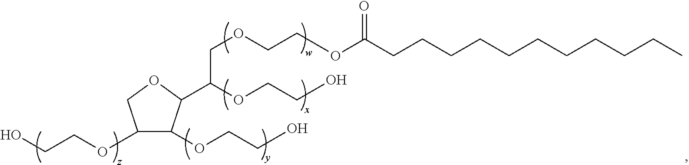

where w, x, y, and z are any suitable positive integers. w, x, y, and z may also each be independently the same or different. In one set of embodiments, w+x+y+z is 20 (e.g., as in Polysorbate 20). In some cases, other polymeric sugars can be used instead of, or in conjunction with, a polysorbate surfactant. Thus, it should be appreciated that, in the specification herein, references to a polysorbate surfactant are by way of example, and in other embodiments, it should be understood that references to a polysorbate surfactant may include other polymeric sugars in conjunction or instead of a polysorbate surfactant.

In some cases, the composition may have a fixed ratio of the stabilization polymer to propylene glycol to the polysorbate surfactant. For instance, the ratio of these may be about 1:1:1, about 1:6:3, about 1:6:2, about 1:7:2, about 1:7:3, about 1.5:1:1, about 1.5:6:3, about 1.5:6:4, about 1:6:2.5, about 1:6.25:2.5, about 1:6.25:2.5, etc. As mentioned above, such ratios may be useful, in certain embodiments of the invention, in providing temperature stability to the composition.

In certain aspects of the invention, a pharmaceutical agent may be combined with a penetrating agent, i.e., an agent that increases transport of the pharmaceutical agent into the skin, relative to transport in the absence of the penetrating agent. In some embodiments, the penetrating agent may define and/or be combined with a hostile biophysical environment. Examples of penetrating agents include oleoresin capsicum or its constituents, or certain molecules containing heterocyclic rings to which are attached hydrocarbon chains.

Non-limiting examples of penetrating agents include, but are not limited to, cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); fatty acid esters (e.g., n-butyrate); organic acids (e.g., citric acid); polyols (e.g., ethylene glycol, glycerol); sulfoxides (e.g., dimethylsulfoxide); terpenes (e.g., cyclohexene); ureas; sugars; carbohydrates or other agents. In certain embodiments, the penetrating agent includes a salt, e.g., as described herein.

In one set of embodiments, pharmaceutical agents are introduced to aid in treatment of medical conditions or diseases, and the symptoms associated thereof. In some embodiments, the invention provides for the treatment of medical conditions or diseases and/or ailments using pharmaceutical agents (for example, to treat a subject diagnosed with a medical condition or disease), and in some cases, the invention provides for the delivery of a minimum amount of pharmaceutical agents to provide effective levels of medication to an affected area topically while limiting side effects. In some cases, the effective dosage of the pharmaceutical agent may be lower than the effective dosage of the pharmaceutical agent when taken orally.

In some embodiments, certain aspects of the invention relate to reducing or avoiding the side effects associated with the delivery of fluticasone and other glucocorticoids orally, nasally, or via inhalation. For example, fluticasone undergoes extensive first-pass metabolism in the liver, and thus has a very short half-life when delivered orally, e.g., about 7.8 hours.

In another aspect, the present invention is directed to a kit including one or more of the compositions discussed herein. A “kit,” as used herein, typically defines a package or an assembly including one or more of the compositions of the invention, and/or other compositions associated with the invention, for example, as described herein. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein, for example, methods of promoting the making or use of compositions such as those discussed above, methods of promoting kits as discussed above, or the like. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

The following documents are incorporated herein by reference: International Patent Application No. PCT/US98/19429, filed Sep. 17, 1998, entitled “A Delivery of Arginine to Cause Beneficial Effects,” by E. T. Fossel, published as WO 99/13717 on Mar. 25, 1999; U.S. patent application Ser. No. 11/587,323, filed Oct. 19, 2006, entitled “Transdermal Delivery of Beneficial Substances Effected by a Hostile Biophysical Environment,” by E. T. Fossel, published as U.S. Patent Application Publication No. 2008/0280984 on Nov. 13, 2008; and U.S. patent application Ser. No. 11/587,328, filed Oct. 19, 2006, entitled “Beneficial Effects of Increasing Local Blood Flow,” by E. T. Fossel, published as U.S. Patent Application Publication No. 2009/0105336 on Apr. 23, 2009.

Also incorporated herein by reference are International Patent Application No. PCT/US2005/005726, filed Feb. 23, 2005, entitled “Topical Delivery of a Nitric Oxide Donor to Improve Body and Skin Appearance,” by E. Fossel, published as WO 2005/081964 on Sep. 9, 2005; International Patent Application No. PCT/US2005/013228, filed Apr. 19, 2005, entitled “Transdermal Delivery of Beneficial Substances Effected by a Hostile Biophysical Environment,” by E. Fossel, published as WO 2005/102282 on Nov. 3, 2005; International Patent Application No. PCT/US2005/013230, filed Apr. 19, 2005, entitled “Beneficial Effects of Increasing Local Blood Flow,” by E. Fossel, published as WO 2005/102307 on Nov. 3, 2005; U.S. patent application Ser. No. 08/932,227, filed Sep. 17, 1997, entitled “Topical Delivery of Arginine of Cause Beneficial Effects,” by E. T. Fossel, published as 2002/0041903 on Apr. 11, 2002; U.S. patent application Ser. No. 10/201,635, filed Jul. 22, 2002, entitled “Topical Delivery of L-Arginine to Cause Beneficial Effects,” by E. T. Fossel, published as 2003/0028169 on Feb. 6, 2003; U.S. patent application Ser. No. 10/213,286, filed Aug. 5, 2002, entitled “Topical and Oral Arginine to Cause Beneficial Effects,” by E. T. Fossel, published as 2003/0018076 on Jan. 23, 2003; U.S. Pat. No. 5,895,658, issued Apr. 20, 1999, entitled “Topical Delivery of L-Arginine to Cause Tissue Warming,” by E. T. Fossel; U.S. Pat. No. 5,922,332, issued Jul. 13, 1999, entitled “Topical Delivery of Arginine to Overcome Pain,” by E. T. Fossel; U.S. Pat. No. 6,207,713, issued Mar. 27, 2001, entitled “Topical and Oral Delivery of Arginine to Cause Beneficial Effects,” by E. T. Fossel; and U.S. Pat. No. 6,458,841, issued Oct. 1, 2002, entitled “Topical and Oral Delivery of Arginine to Cause Beneficial Effects,” by E. T. Fossel.

The following examples are intended to illustrate certain embodiments of the present invention, but do not exemplify the full scope of the invention.

### Example 1

This example illustrates the preparation of compositions in accordance with certain embodiments of the invention. The ingredients are shown in Table 1, along with the order of addition of the ingredients (ingredients with the same numbers were added at or nearly at the same time). It should also be appreciated that the relative amounts of each component may be varied (e.g., by about +/−15% or about +/−10%) in some embodiments. Those of ordinary skill in the art will also understand that percentages other than the ones listed below are also possible, according to other embodiments of the invention.

TABLE 1

| Ingredient | Order | % by Weight |
|---|---|---|
| Distilled water | 1 | 43.59% |
| Sodium chloride | 2 | 10.68% |
| Potassium chloride | 3 | 5.34% |
| L-arginine HCl | 4 | 8.01% |
| Propylene glycol | 5 | 5.34% |
| Xanthan gum | 5 | 0.85% |
| Glyceryl stearate | 1 | 7.47% |
| Cetyl alcohol (1-hexadecanol) | 1 | 7.47% |
| Polysorbate 20 | 2 | 2.14% |
| Isopropyl myristate | 2 | 1.07% |
| Oleic acid | 2 | 1.07% |
| Squalane | 2 | 4.27% |
| Fluticasone propionate | 3 | 2.50% |

The ingredients were added with continuous overhead stirring of the mixture. The mixture was heated to 70° C. and xanthan gum was added while being mixed at high speed to ensure hydration of the xanthan gum. The lipid ingredients were heated to liquefaction and the fluticasone added while mixing. After reaching 70° C., these ingredients were added to the mixture. The mixture was then mixed and allowed to cooled to ambient temperatures.

### Example 2

This example is similar to Example 1, except slightly different amounts of the ingredients were used, as can be seen in Table 2. The method for making the final formulation is the same as in Example 1.

TABLE 2

| Ingredient | Order | % by Weight |
|---|---|---|
| Distilled water | 1 | 44.33% |
| Sodium chloride | 2 | 10.87% |
| Potassium chloride | 3 | 5.43% |
| L-arginine HCl | 4 | 8.15% |
| Propylene glycol | 5 | 5.34% |
| Xanthan gum | 5 | 0.86% |
| Glyceryl stearate | 1 | 7.59% |
| Cetyl alcohol (1-hexadecanol) | 1 | 7.59% |
| Polysorbate 20 | 2 | 2.18% |
| Isopropyl myristate | 2 | 1.09% |
| Oleic acid | 2 | 1.09% |
| Squalane | 2 | 4.34% |
| Fluticasone propionate | 3 | 1.00% |

### Example 3

In this example, a 71 year-old male with severe arthritis pain in the top of the right foot applied 1 g of 1.5% fluticasone cream (see Example 1) every 4 hours for 12 hours. After that, the pain was completely gone and remained absent for about three weeks. After three weeks, the cream was then re-applied (every 4 hours for 12 hours) with similar results. This process was repeated every 3 to 4 weeks to maintain pain relief.

### Example 4

In this example, a 77 year-old woman with limited neck mobility and neck pain due to a disc abnormality applied 1 g of 1.5% fluticasone cream (see Example 1) twice, with 4 hours in between applications. Within 3 to 4 hours, after the second application, her neck was fully mobile and the pain was absent. This relief persisted for several weeks.

### Example 5

In this example, an 84 year-old male was bitten multiple times by mosquitos. He received 4 bites. He applied 0.1 g of fluticasone cream (see Example 1) to each of two bites and left two bites untreated. Within an hour, any sign of the treated bites and the corresponding itch was absent. However, the untreated bites remained itchy and red for 2-3 days afterwards.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition for transdermal delivery, the composition comprising:
an ionic salt at a concentration of at least about 5 wt %; and
fluticasone and/or a salt thereof.

2. The composition of claim 1, wherein the ionic salt, the nitric oxide donor, and the fluticasone and/or the salt thereof are contained within a delivery vehicle.

3. The composition of claim 2, wherein the delivery vehicle is a cream.

4. The composition of claim 2, wherein the delivery vehicle is a gel.

5. The composition of claim 2, wherein the delivery vehicle is a lotion.

6. The composition of claim 2, wherein the delivery vehicle is contained within a transdermal patch.

7. The composition of claim 1, wherein the composition further comprises a nitric oxide donor.

8. The composition of claim 7, wherein the nitric oxide donor comprises L-arginine.

9. The composition of claim 7, wherein the nitric oxide donor comprises an L-arginine salt.

10. The composition of claim 7, wherein the nitric oxide donor comprises L-arginine HCl.

11. The composition of claim 7 wherein the nitric oxide donor is present at a concentration of at least about 0.5% by weight of the composition.

12. The composition of claim 7, wherein the nitric oxide donor is present at a concentration of at least about 5% by weight of the composition.

13. (canceled)

14. The composition of claim 1, wherein the ionic salt comprises sodium chloride.

15. The composition of claim 1, wherein the ionic salt comprises sodium citrate.

16. The composition of claim 1, wherein composition has an ionic strength of at least about 0.25 M.

17. The composition of claim 1, wherein composition has an ionic strength of at least about 1 M.

18. The composition of claim 1, wherein the subject is human.

19. The composition of claim 1, wherein the composition further comprises xanthan gum.

20. The composition of claim 1, wherein the composition further comprises a polysorbate.

21. (canceled)

22. The composition of claim 1, wherein the fluticasone and/or the salt thereof is present at a concentration of at least about 0.025% by weight of the composition.

23. The composition of claim 1, wherein the fluticasone and/or the salt thereof is present at a concentration of at least about 1% by weight of the composition.

24. (canceled)

25. A method, comprising applying the composition of claim 1 to a subject.

26. A composition for transdermal delivery, the composition comprising:
an ionic salt at a concentration of at least about 5 wt %; and
a glucocorticoid and/or salt thereof.

27. The composition of claim 26, wherein the glucocorticoid and/or salt thereof is selected from the group consisting of beclomethasone, budesonide, fluticasone, mometasone and ciclesonide, and salts thereof.

28. The composition of claim 26, wherein the composition further comprises a nitric oxide donor.

29-34. (canceled)

35. A composition for transdermal delivery, wherein at least about 80% by weight of the composition comprises:
an ionic salt at a concentration of at least about 5 wt %;
glucocorticoid and/or salt thereof.

36-98. (canceled)
